(19) United States
(12) Patent Application Publication
(10) Pub. No.: US 2021/0132362 A1
(43) Pub. Date: May 6, 2021

(54) IMAGE PICKUP APPARATUS, ENDOSCOPE, ENDOSCOPE SYSTEM, AND DRIVING METHOD FOR IMAGE PICKUP APPARATUS

(71) Applicant: OLYMPUS CORPORATION, Tokyo (JP)

(72) Inventor: Susumu YAMAZAKI, Tachikawa-shi (JP)

(73) Assignee: OLYMPUS CORPORATION, Tokyo (JP)

(21) Appl. No.: 17/149,906

(22) Filed: Jan. 15, 2021

Related U.S. Application Data
(63) Continuation of application No. PCT/JP2019/030509, filed on Aug. 2, 2019.

(30) Foreign Application Priority Data
Aug. 8, 2018 (JP) 2018-149380

(51) Int. Cl. G02B 23/24; A61B 1/00; H04N 5/3745

(52) U.S. Cl. CPC G02B 23/2484; A61B 1/00027; H04N 2005/2255; H04N 5/3745; A61B 1/00006

(57) ABSTRACT

An image pickup apparatus includes: an image pickup device including a plurality of pixels generating an image pickup signal corresponding to a light reception amount; a universal cable for transmitting electric power to the image pickup device; an AC-voltage-pulse-signal generation circuit provided on a proximal end side of the universal cable and generating an AC voltage pulse signal obtained by converting a positive voltage level and a negative voltage level of an inputted pulse signal into a predetermined positive voltage level and a predetermined negative voltage level, respectively, and outputting the AC voltage pulse signal to the universal cable; and a voltage adjustment circuit provided on a distal end side of the universal cable and converting the predetermined positive voltage level and the predetermined negative voltage level of the AC voltage pulse signal transmitted from the universal cable into a DC voltage level and output a DC voltage pulse signal.

### CROSS REFERENCE TO RELATED APPLICATION

This application is a continuation application of PCT/JP2019/030509 filed on Aug. 2, 2019 and claims benefit of Japanese Application No. 2018-149380 filed in Japan on Aug. 8, 2018, the entire contents of which are incorporated herein by this reference.

### BACKGROUND OF THE INVENTION

### 1. Field of the Invention

The present invention relates to an image pickup apparatus that picks up an image of an object and generates image data of the object, an endoscope, an endoscope system, and a driving method for the image pickup apparatus.

### 2. Description of the Related Art

An endoscope system including an endoscope that picks up an image of an object on an inside of a subject and a processor that generates an observation image of the object, the image of which is picked up by the endoscope, have been widely used in a medical field, an industrial field, and the like.

For example, Japanese Patent No. 6138406 discloses an image pickup apparatus that separates, with a separating unit and a pulse-signal detecting unit, a negative voltage pulse signal generated by a pulse-signal superimposing unit of a connector unit respectively into a negative voltage (negative power) and a pulse signal and outputs the negative voltage and the pulse signal to a first chip.

An image pickup unit mounted on a distal end portion of an insertion section of an endoscope is desirably small in a chip area and small in size for a reduction in a diameter of the insertion section of the endoscope. Therefore, it is desired to integrate the pulse-signal detecting unit on a chip and reduce the image pickup unit in size.

### SUMMARY OF THE INVENTION

An image pickup apparatus according to an aspect of the present invention includes: an image pickup device including a plurality of pixels that are disposed in a two-dimensional matrix shape, the plurality of pixels being configured to receive light from an outside and generate an image pickup signal corresponding to a light reception amount; a transmission cable for transmitting electric power to the image pickup device; an AC-voltage-pulse-signal generation circuit provided on a proximal end side of the transmission cable, the AC-voltage-pulse-signal generation circuit being configured to generate an AC voltage pulse signal obtained by converting a positive voltage level and a negative voltage level of an inputted pulse signal into a predetermined positive voltage level and a predetermined negative voltage level, respectively, and output the AC voltage pulse signal to the transmission cable; and a voltage adjustment circuit provided on a distal end side of the transmission cable, the voltage adjustment circuit being configured to convert the predetermined positive voltage level and the predetermined negative voltage level of the AC voltage pulse signal transmitted from the transmission cable into a DC voltage level and output a DC voltage pulse signal.

An endoscope according to an aspect of the present invention includes: the image pickup apparatus according to the aspect described above; an insertion section insertable into a subject; and a connector detachably attached to an image processing apparatus that applies image processing to the image pickup signal. The image pickup device and the voltage adjustment circuit are provided on a distal end side of the insertion section. The AC-voltage-pulse-signal generation circuit is provided in the connector.

An endoscope system according to an aspect of the present invention includes: the endoscope according to the aspect described above; and an image processing apparatus configured to apply image processing to the image pickup signal.

A driving method for an image pickup apparatus according to an aspect of the present invention is a driving method for an image pickup apparatus configured from: an image pickup device including a plurality of pixels that are disposed in a two-dimensional matrix shape, the plurality of pixels being configured to receive light from an outside and generate an image pickup signal corresponding to a light reception amount; and a transmission cable for transmitting electric power to the image pickup device, the driving method for the image pickup apparatus including: generating, with an AC-voltage-pulse-signal generation circuit provided on a proximal end side of the transmission cable, an AC voltage pulse signal obtained by converting a positive voltage level and a negative voltage level of an inputted pulse signal into a predetermined positive voltage level and a predetermined negative voltage level, respectively, and outputting the AC voltage pulse signal to the transmission cable;

converting, with a voltage adjustment circuit provided on a distal end side of the transmission cable, the predetermined positive voltage level and the predetermined negative voltage level of the AC voltage pulse signal transmitted from the transmission cable into a DC voltage level and outputting a DC voltage pulse signal; and driving the image pickup device using the DC voltage pulse signal.

### BRIEF DESCRIPTION OF THE DRAWINGS

FIG. 1 is an overall configuration diagram showing an example of an overall configuration of an endoscope system according to a first embodiment;

FIG. 2 is a block diagram showing a configuration of a main part of the endoscope system 1 according to the first embodiment;

FIG. 3 is a circuit diagram showing an example of a configuration of a voltage adjusting unit 39;

FIG. 4 is a timing chart showing an example of operation of the endoscope system according to the first embodiment;

FIG. 5 is a circuit diagram showing another example of the configuration of the voltage adjusting unit 39;

FIG. 6 is a circuit diagram showing another example of the configuration of the voltage adjusting unit 39;

FIG. 7 is a circuit diagram showing another example of the configuration of the voltage adjusting unit 39;

FIG. 8 is a circuit diagram showing another example of the configuration of the voltage adjusting unit 39;

FIG. 9 is a block diagram showing a configuration of a main part of an endoscope system 1 according to a second embodiment;

FIG. 10 is a circuit diagram showing an example of a configuration of a voltage adjusting unit 39A;

FIG. 11 is a timing chart showing an example of operation of the endoscope system according to the second embodiment;

FIG. 12 is a block diagram showing a configuration of a main part of an endoscope system 1 according to a third embodiment;

FIG. 13 is a circuit diagram showing an example of a configuration of a voltage adjusting unit 39B; and

FIG. 14 is a timing chart showing an example of operation of the endoscope system according to the third embodiment.

### DETAILED DESCRIPTION OF THE PREFERRED EMBODIMENTS

Embodiments of the present invention are explained below with reference to the drawings.

### First Embodiment

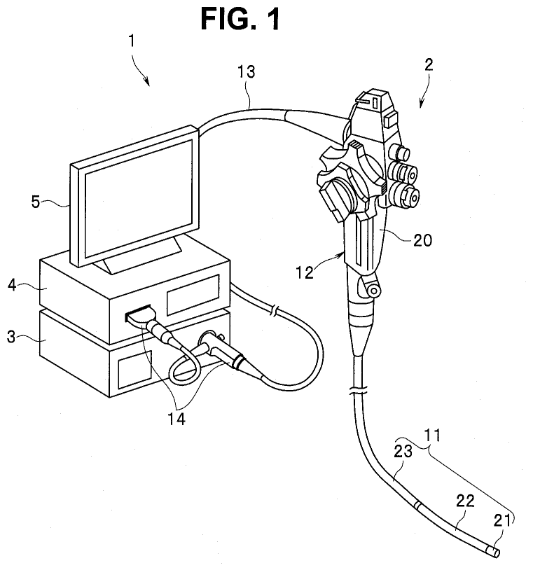

FIG. 1 is an overall configuration diagram showing an example of an overall configuration of an endoscope system according to a first embodiment.

As shown in FIG. 1, a main part of an endoscope system 1 includes an endoscope 2, a light source apparatus 3, a video processor 4 functioning as an image processing apparatus, and a display apparatus 5.

The endoscope 2 includes an elongated insertion section 11 to be inserted into an observation target part of a subject, an operation unit 12 connected consecutively to a proximal end portion of the insertion section 11, a universal cable 13 extended from a side surface of the operation unit 12, and a connector unit 14 provided at an extension end portion of the universal cable 13. The connector unit 14 includes a light source connector, an electric cable extended from a side portion of the light source connector, and an electric connector disposed at an extension end of the electric cable. Note that the light source connector of the connector unit 14 is detachably connected to the light source apparatus 3. The electric connector of the connector unit 14 is detachably connected to the video processor 4.

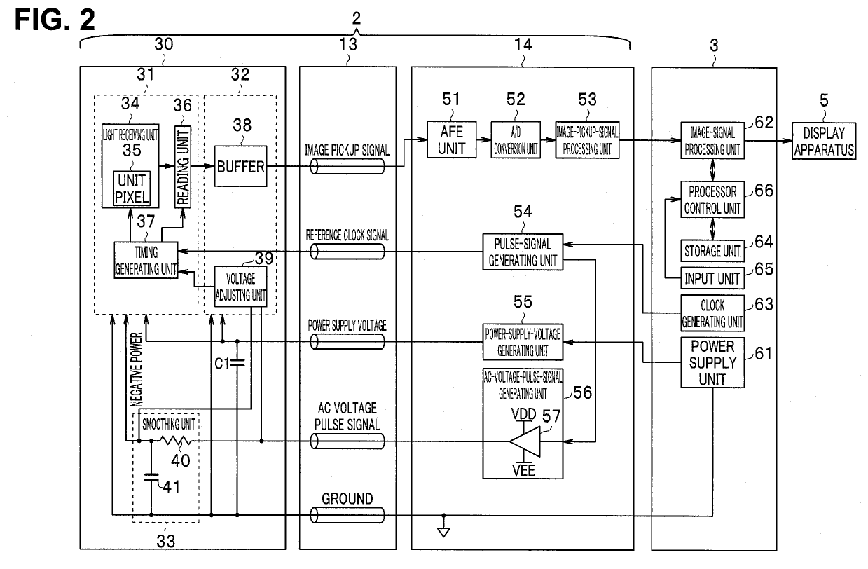

The insertion section 11 includes a distal end portion 21 on a distal end side. A bendable bending section 22 is connected consecutively to a proximal end portion of the distal end portion 21. Further, a long flexible tube section 23 having flexibility formed by a soft tubular member is connected consecutively to a proximal end portion of the bending section 22. An image pickup unit 30 (see FIG. 2) for acquiring image information of a subject is provided at the distal end portion 21.

The operation unit 12 includes an operation unit main body 20 configuring an operation grasping section. In the operation unit main body 20, an angle knob for bending the bending section 22 of the insertion section 11 is turnably disposed and a suction button, an air/water feeding button, switches for various endoscopic functions, and the like are provided.

The light source apparatus 3 supplies illumination light to a light guide (not illustrated) provided in the endoscope 2. In other words, the light guide is disposed in the universal cable 13, the operation unit 12, and the insertion section 11 of the endoscope 2 in the present embodiment. The light source apparatus 3 supplies, via the light guide, the illumination light to an illumination optical system configuring an illumination window of the distal end portion 21. The illumination light is diverged by the illumination optical system and irradiates a site to be examined.

The video processor 4 applies image processing to image data picked up by the endoscope 2 to generate an image signal and outputs the generated image signal to the display apparatus 5. The display apparatus 5 displays an image corresponding to the image signal generated by the video processor 4.

The video processor 4 performs control of the entire endoscope system 1. For example, the video processor 4 performs control for switching illumination light emitted by the light source apparatus 3 and switching an image pickup mode of the endoscope 2.

FIG. 2 is a block diagram showing a configuration of a main part of the endoscope system 1 in the first embodiment. Details of various unit configurations of the endoscope system 1 and a route of an electric signal in the endoscope system 1 are explained with reference to FIG. 2.

First, a configuration of the endoscope 2 is explained. The endoscope 2 functioning as an image pickup apparatus shown in FIG. 2 includes the image pickup unit 30, the universal cable 13 configuring a transmission cable, and the connector unit 14.

The image pickup unit 30 includes a first chip 31, a second chip 32, and a smoothing unit 33. A capacitor C1 for power stabilization is provided between a power supply voltage VDD and a ground GND supplied to the image pickup unit 30.

The first chip 31 functioning as an image pickup device includes a light receiving unit 34 in which a plurality of unit pixels 35 that are disposed in a two-dimensional matrix shape and receive light from an outside and generate and output an image pickup signal corresponding to a light reception amount are disposed, a reading unit 36 that reads an image pickup signal photoelectrically converted in each of the plurality of unit pixels 35 in the light receiving unit 34, and a timing generating unit 37 that generates, based on a reference clock signal received from the connector unit 14 and a pulse signal received from a voltage adjusting unit 39 explained below, driving signals including a light receiving unit driving signal for driving the light receiving unit 34 and a reading unit driving signal for driving the reading unit 36 and outputs the driving signals to the light receiving unit 34 and the reading unit 36.

The second chip 32 includes a buffer 38 that amplifies the image pickup signal outputted from each of the plurality of unit pixels 35 in the first chip 31 and outputs the image pickup signal to the universal cable 13 and a voltage adjusting unit 39 that converts an AC voltage pulse signal from an AC-voltage-pulse-signal generating unit 56 explained below into a DC voltage level, a High side of which is set to 3 V (VDD=3V) and a Low side of which is set to 0 V (a ground GND), and outputs a pulse signal of a DV voltage (a DC voltage pulse signal) to the timing generating unit 37.

The smoothing unit 33 configuring a smoothing circuit is connected between the first chip 31 and the universal cable 13 and between the second chip 32 and the universal cable 13. The smoothing unit 33 separates a DC component and an AC component from a negative voltage transmitted from the universal cable 13 and outputs the separated DC component to the first chip 31. The smoothing unit 33 includes a resistor 40 (for example, 100Ω) connected in series to the universal cable 13 (a signal line) through which the negative voltage explained below is transmitted and a bypass capacitor 41 connected between the AC-voltage-pulse-signal generating unit 56 explained below and the ground GND. The smoothing unit 33 forms an RC circuit (a low-pass filter circuit). Consequently, a pulse signal of the AC component superimposed on the negative voltage received from the connector unit 14 explained below is cut and the DC component is outputted to the unit pixels 35.

The universal cable 13 is configured using at least five signal lines, that is, a signal line for transmitting a power supply voltage generated by a power-supply-voltage generating unit 55 to the image pickup unit 30, a signal line for transmitting an AC voltage pulse signal generated by the AC-voltage-pulse-signal generating unit 56 to the image pickup unit 30, a signal line for transmitting a reference clock signal generated by a pulse-signal generating unit 54 to the image pickup unit 30, a signal line for transmitting an image pickup signal generated by the image pickup unit 30 to the connector unit 14, and a signal line for transmitting the ground GND to the image pickup unit 30.

The connector unit 14 includes an analog front end unit 51 (hereinafter referred to as “AFE unit 51”), an A/D conversion unit 52, an image-pickup-signal processing unit 53, a pulse-signal generating unit 54, a power-supply-voltage generating unit 55, and an AC-voltage-pulse-signal generating unit 56.

The AFE unit 51 receives an image pickup signal propagated from the image pickup unit 30 and, after performing impedance matching using a passive element such as a resistor, extracts an AC component using a capacitor and determines an operating point according to partial resistance. Thereafter, the AFE unit 51 amplifies the image pickup signal (an analog signal) and outputs the image pickup signal to the A/D conversion unit 52.

The A/D conversion unit 52 converts the analog image pickup signal received from the AFE unit 51 into a digital image pickup signal and outputs the digital image pickup signal to the image-pickup-signal processing unit 53.

The image-pickup-signal processing unit 53 is configured by, for example, an FPGA (field programmable gate array). The image-pickup-signal processing unit 53 performs processing such as noise removal and format conversion processing on the digital image pickup signal received from the A/D conversion unit 52 and outputs the processed digital image pickup signal to the video processor 4.

The pulse-signal generating unit 54 generates, based on a clock signal (for example, a 27 MHz clock signal) supplied from the video processor 4 and serving as a reference for operation of the respective components of the endoscope 2, a reference clock signal serving as a reference for operation of the respective components of the image pickup unit 30 and outputs the reference clock signal to the timing generating unit 37 of the image pickup unit 30 via the universal cable 13. The pulse-signal generating unit 54 outputs, to the AC-voltage-pulse-signal generating unit 56, a pulse signal for generating a driving signal for the image pickup unit 30 based on the clock signal supplied from the video processor 4 and serving as the reference for the operation of the respective components of the endoscope 2.

The power-supply-voltage generating unit 55 is provided on a proximal end side of the universal cable 13. The power-supply-voltage generating unit 55 generates, from power supplied from the video processor 4, a power supply voltage VDD necessary for driving the first chip 31 and the second chip 32 and outputs the power supply voltage VDD to the first chip 31 and the second chip 32. The power-supply-voltage generating unit 55 generates, using a regulator or the like, the power supply voltage VDD necessary for driving the first chip 31 and the second chip 32.

The AC-voltage-pulse-signal generating unit 56 configuring an AC-voltage-pulse-signal generation circuit includes a buffer amplifier 57 driven by positive power (VDD=4.5V) having a predetermined positive voltage level and negative power (VEE=−1V) having a predetermined negative voltage level. The buffer amplifier 57 converts a positive voltage level of a pulse signal supplied from the pulse-signal generating unit 54 into 4.5 V with the positive power and converts a negative voltage level of the pulse signal into −1 V with the negative power. In other words, the AC-voltage-pulse-signal generating unit 56 is provided on the proximal end side of the universal cable 13 and generates an AC voltage pulse signal, a High side of which is 4.5 V and a Low side of which is −1 V, based on the pulse signal supplied from the pulse-signal generating unit 54 and outputs the AC voltage pulse signal to the image pickup unit 30 via the universal cable 13.

A configuration of the video processor 4 is explained.

The video processor 4 is a control apparatus that collectively controls the entire endoscope system 1. The video processor 4 includes a power supply unit 61, an image-signal processing unit 62, a clock generating unit 63, a storage unit 64, an input unit 65, and a processor control unit 66.

The power supply unit 61 generates a power supply voltage and supplies the generated power supply voltage to the power-supply-voltage generating unit 55 of the connector unit 14 together with the ground (GND).

The image-signal processing unit 62 performs image processing such as synchronization processing, white balance (WB) adjustment processing, gain adjustment processing, gamma correction processing, digital analog (D/A) 39204Z conversion processing, and format conversion processing on the digital image pickup signal subjected to the signal processing by the image-pickup-signal processing unit 53 to convert the digital image pickup signal into an image signal and outputs the image signal to the display apparatus 5.

The clock generating unit 63 generates a clock signal serving as a reference for operation of the respective components of the endoscope system 1 and outputs the clock signal to the pulse-signal generating unit 54.

The storage unit 64 stores various kinds of information concerning the endoscope system 1, data being processed, and the like. The storage unit 64 is configured using a storage medium such as a Flash memory or a RAM (random access memory).

The input unit 65 receives inputs of various kinds of operation concerning the endoscope system 1. For example, the input unit 65 receives an instruction signal for switching a type of illumination light emitted by the light source apparatus 3. The input unit 65 is configured using, for example, a cross switch or a push button.

The processor control unit 66 collectively controls the respective units configuring the endoscope system 1. The processor control unit 66 is configured using a CPU (central processing unit) or the like. The processor control unit 66 switches, according to an instruction signal received from the input unit 65, illumination light emitted by the light source apparatus 3.

By configuring the image pickup unit 30 in this way, a negative voltage supplied from the AC-voltage-pulse-signal generating unit 56 is used for driving of the unit pixels 35. Since a required electric current is small, it is possible to supply a voltage from the bypass capacitor 41 of the smoothing unit 33 if supplied for a short time. In the smoothing unit 33, an RC circuit (a low-pass filter circuit) is formed using the bypass capacitor 41 and the resistor 40, whereby a pulse signal is sufficiently reduced and transmitted to the unit pixels 35. Further, the voltage adjusting unit 39 configuring a voltage adjustment circuit generates a pulse signal of a DC voltage (a DC voltage pulse signal), a High side of which is set to 3 V (VDD=3V) and a Low side of which is set to 0 V (the ground GND), and outputs the pulse signal to the timing generating unit 37.

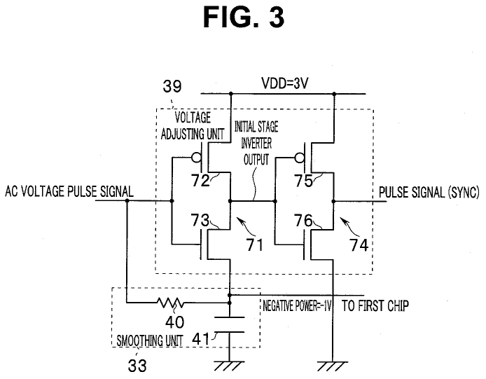

A detailed configuration of the voltage adjusting unit 39 is explained. FIG. 3 is a circuit diagram showing an example of a configuration of the voltage adjusting unit 39.

As shown in FIG. 3, the voltage adjusting unit 39 includes an inverter circuit 71 in an initial stage (a pre-stage) configured by a PMOS transistor 72 and an NMOS transistor 73 and an inverter circuit 74 in a post-stage configured by a PMOS transistor 75 and an NMOS transistor 76. Note that the voltage adjusting unit 39 is configured using normal inverter circuits 71 and 74. However, the voltage adjusting unit 39 is not limited to this. For example, as a countermeasure for noise, the voltage adjusting unit 39 may be configured using an inverter circuit having a hysteresis characteristic.

The voltage adjusting unit 39 has a configuration in which the two inverter circuits 71 and 74 are connected in series. An output terminal of the inverter circuit 71 in the initial stage is connected to an input terminal of the inverter circuit 74 in the post-stage.

An output terminal of the AC-voltage-pulse-signal generating unit 56 is connected to an input terminal of the inverter circuit 71 in the initial stage to which a gate terminal of the PMOS transistor 72 and a gate terminal of the NMOS transistor 73 are connected. An AC voltage pulse signal is inputted to the input terminal of the inverter circuit 71 in the initial stage from the AC-voltage-pulse-signal generating unit 56.

A source terminal of the PMOS transistor 72 of the inverter circuit 71 in the initial stage is connected to a power supply (VDD=3V). A source terminal of the NMOS transistor 73 is connected to an output (negative power=−1V) of the smoothing unit 33. Further, a drain terminal of the PMOS transistor 72 and a drain terminal of the NMOS transistor 73 of the inverter circuit 71 in the initial stage are connected to configure an output terminal.

The output terminal of the inverter circuit 71 in the initial stage is connected to the input terminal of the inverter circuit 74 in the post-stage to which a gate terminal of the PMOS transistor 75 and a gate terminal of the NMOS transistor 76 are connected.

A source terminal of the PMOS transistor 75 of the inverter circuit 74 in the post-stage is connected to the power supply (VDD=3V). A source terminal of the NMOS transistor 76 is connected to the ground GND (0V). Further, a drain terminal of the PMOS transistor 75 and a drain terminal of the NMOS transistor 76 of the inverter circuit 74 in the post-stage are connected to configure an output terminal. The output terminal of the inverter circuit 74 in the post-stage is connected to the timing generating unit 37. A pulse signal outputted from the inverter circuit 74 in the post-stage is inputted to the timing generating unit 37.

Like the voltage adjusting unit 39 in the present embodiment, the pulse-signal detecting unit disclosed in Japanese Patent No. 6138406 is configured to output a pulse signal. However, the pulse-signal detecting unit is configured by a high-pass filter. When the pulse-signal detecting unit configured by the high-pass filter having a large time constant is incorporated in, for example, the second chip 32 in the present embodiment, an area of the second chip 32 increases.

In contrast, the voltage adjusting unit 39 in the present embodiment has a configuration in which two logic circuits are combined. Therefore, when the voltage adjusting unit 39 is incorporated in the second chip 32, the area of the second chip 32 can be reduced more than when the high-pass filter having the large time constant is incorporated in the second chip 32.

Note that, in the present embodiment, the voltage adjusting unit 39 is provided in the second chip 32. However, the voltage adjusting unit 39 is not limited to this and may be provided in the first chip 31. In the present embodiment, the image pickup unit 30 includes the two chips, that is, the first chip 31 and the second chip 32. However, the image pickup unit 30 is not limited to this and may include, for example, one chip including respective circuits of the first chip 31 and respective circuits of the second chip 32.

Operation of the endoscope system 1 in the first embodiment configured as explained above is explained.

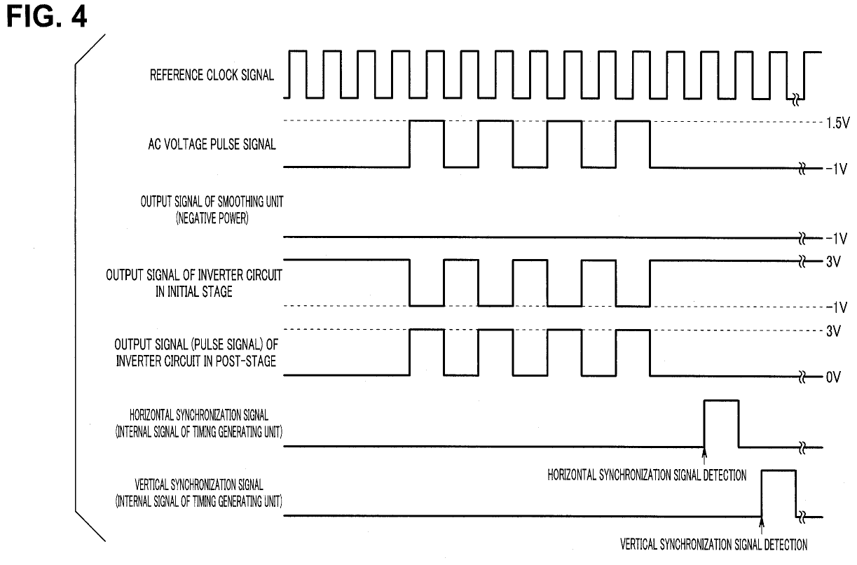

FIG. 4 is a timing chart showing an example of operation of the endoscope system according to the first embodiment. In FIG. 4, a reference clock signal, an AC voltage pulse signal, an output signal of the smoothing unit 33, an output signal of the inverter circuit 71 in the initial stage, an output signal of the inverter circuit 74 in the post-stage, a horizontal synchronization signal, and a vertical synchronization signal are shown in order from a top.

An AC voltage pulse signal, a High side of which is 4.5 V and a Low side of which is −1 V, is generated by the buffer amplifier 57 of the AC-voltage-pulse-signal generating unit 56 and outputted to the smoothing unit 33 and the voltage adjusting unit 39. However, the High side of the AC voltage pulse signal outputted from the AC-Voltage-pulse-signal generating unit 56 drops to 1.5 V because of attenuation in the universal cable 13. Accordingly, as shown in FIG. 4, an AC voltage pulse signal, a High side of which is 1.5 V and a Low side of which is −1 V, is inputted to the smoothing unit 33 and the voltage adjusting unit 39.

The smoothing unit 33 smooths the AC voltage pulse signal by the low-pass filter circuit formed by the resistor 40 and the bypass capacitor 41 and generates and outputs a constant voltage (negative power) of −1 V.

A threshold of the inverter circuit 71 in the initial stage of the voltage adjusting unit 39 is 1 V. The source terminal of the PMOS transistor 72 of the inverter circuit 71 is connected to the power supply (VDD=3V) and the source terminal of the NMOS transistor 73 is connected to an output (negative power=−1V) of the smoothing unit 33.

Accordingly, the inverter circuit 71 in the initial stage of the voltage adjusting unit 39 outputs an output signal of −1 V when the AC voltage pulse signal is 1.5 V and outputs an output signal of 3 V when the AC voltage pulse signal is −1 V.

A threshold of the inverter circuit 74 in the post-stage of the voltage adjusting unit 39 is 1.5 V. The source terminal of the PMOS transistor 75 of the inverter circuit 74 is connected to the power supply (VDD=3V) and the source terminal of the NMOS transistor 76 is connected to the ground GND (0V).

Accordingly, the inverter circuit 74 in the post-stage of the voltage adjusting unit 39 outputs an output signal of 0 V when the output signal of the inverter circuit 71 in the pre-stage is 3 V and outputs an output signal of 3 V when the output signal of the inverter circuit 71 in the pre-stage is −1 V.

Consequently, the voltage adjusting unit 39 outputs a pulse signal, a High side of which is 3 V and a Low side of which is 0 V, to the timing generating unit 37. The timing generating unit 37 configuring a timing generation circuit generates a horizontal synchronization signal and a vertical synchronization signal based on the reference clock signal received from the pulse-signal generating unit 54 and the pulse signal received from the voltage adjusting unit 39. The timing generating unit 37 generates a driving signal based on the generated horizontal synchronization signal and the generated vertical synchronization signal and outputs the driving signal to the light receiving unit 34 and the reading unit 36.

As explained above, the voltage adjusting unit 39 is configured by the two inverter circuits 71 and 74, that is, two logic circuits. Therefore, when the voltage adjusting unit 39 is provided in the second chip 32, a chip area can be set smaller than when the high-pass filter having the large time constant is provided.

Accordingly, with the endoscope 2 functioning as the image pickup apparatus in the present embodiment, even when a circuit that outputs a pulse signal is mounted on a chip, a chip area can be reduced.

### (Modification 1 of the First Embodiment)

A modification 1 of the first embodiment is explained.

The voltage adjusting unit 39 in the first embodiment explained above includes the two inverter circuits 71 and 74 as the two logic circuits. However, the voltage adjusting unit 39 may have another configuration.

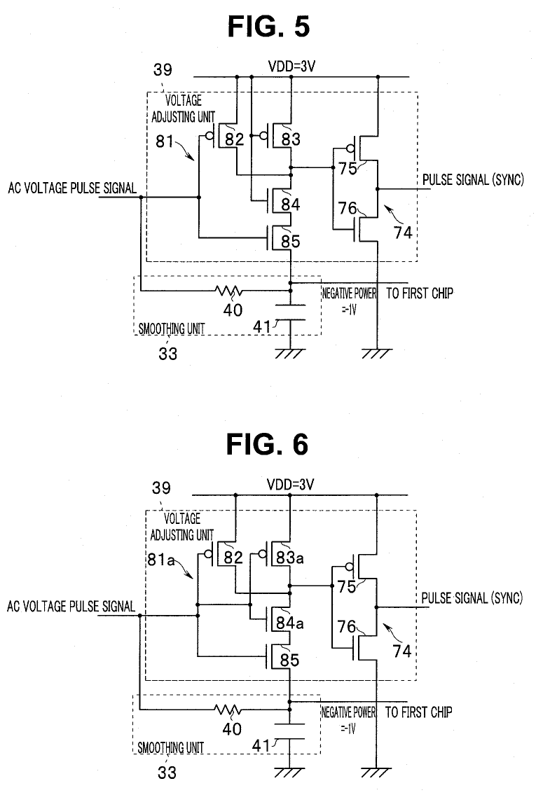

FIG. 5 is a circuit diagram showing another example of the configuration of the voltage adjusting unit 39. Note that, in FIG. 5, the same components as the components shown in FIG. 3 are denoted by the same reference numerals and signs and explanation of the components is omitted.

As shown in FIG. 5, the voltage adjusting unit 39 is configured by a NAND circuit 81 in a pre-stage and the inverter circuit 74 in the post-stage. The NAND circuit 81 is a two-input NAND gate and includes PMOS transistors 82 and 83 and NMOS transistors 84 and 85.

An AC voltage pulse signal is inputted to one input terminal of the NAND circuit 81 and power (VDD=3V) is inputted to the other input terminal. More specifically, the AC voltage pulse signal is inputted to a gate terminal of the PMOS transistor 82 and a gate terminal of the NMOS transistor 85 and the power (VDD=3V) is inputted to a gate terminal of the PMOS transistor 83 and a gate terminal of the NMOS transistor 84.

The NAND circuit 81 outputs an L signal when an H signal is inputted to the two input terminals and outputs the H signal when other signals are inputted to the two input terminals. Accordingly, when 1.5 V is inputted to one input terminal as an AC voltage pulse signal and power (3V) is inputted to the other input terminal, the NAND circuit 81 outputs −1 V, which is an output of the smoothing unit 33. When −1 V is inputted to one input terminal as the AC voltage pulse signal and the power (3V) is inputted to the other input terminal, the NAND circuit 81 outputs 3 V.

The inverter circuit 74 in the post-stage has the same configuration as the configuration in the first embodiment. The inverter circuit 74 in the post-stage outputs an output signal of 0 V when an output signal of the NAND circuit 81 in the pre-stage is 3 V and outputs an output signal of 3 V when the output signal of the NAND circuit 81 in the pre-stage is −1 V. Consequently, as in the first embodiment, the voltage adjusting unit 39 can output a pulse signal, a High side of which is 3 V and a Low side of which is 0 V, to the timing generating unit 37.

In this way, the voltage adjusting unit 39 is configured by two logic circuits, that is, the NAND circuit 81 in the pre-stage and the inverter circuit 74 in the post-stage. Therefore, as in the first embodiment, a chip area can be set smaller than when the high-pass filter having the large time constant is provided.

FIG. 6 is a circuit diagram showing another example of the configuration of the voltage adjusting unit 39. Note that, in FIG. 6, the same components as the components shown in FIG. 5 are denoted by the same reference numerals and signs and explanation of the components is omitted.

As shown in FIG. 6, the voltage adjusting unit 39 is configured using a NAND circuit 81a instead of the NAND circuit 81 shown in FIG. 5. The NAND circuit 81a is configured using a PMOS transistor 83a and an NMOS transistor 84a respectively instead of the PMOS transistor 83 and the NMOS transistor 84 shown in FIG. 5.

An AC voltage pulse signal is inputted to a gate terminal of the PMOS transistor 83a and a gate terminal of the NMOS transistor 84a. The other components are the same as the components shown in FIG. 5. In other words, two input terminals of the NAND circuit 81a are shared. The AC voltage pulse signal is inputted to both of the two input terminals.

Accordingly, when 1.5 V is inputted to the two input terminals as the AC voltage pulse signal, the NAND circuit 81a outputs −1 V, which is an output of the smoothing unit 33. When −1 V is inputted to the two input terminals as the AC voltage pulse signal, the NAND circuit 81a outputs 3 V.

The inverter circuit 74 in the post-stage has the same configuration as the configuration in the first embodiment. The inverter circuit 74 in the post-stage outputs an output signal of 0 V when an output signal of the NAND circuit 81a in the pre-stage is 3 V and outputs an output signal of 3 V when the output signal of the NAND circuit 81a in the pre-stage is −1 V. Consequently, as in the first embodiment, the voltage adjusting unit 39 can output a pulse signal, a High side of which is 3 V and a Low side of which is 0 V, to the timing generating unit 37.

In this way, the voltage adjusting unit 39 is configured by two logic circuits, that is, the NAND circuit 81a in the pre-stage and the inverter circuit 74 in the post-stage. Therefore, as in the first embodiment, a chip area can be set smaller than when the high-pass filter having the large time constant is provided.

Accordingly, with the endoscope 2 functioning as an image pickup apparatus in the modification 1, as in the endoscope 2 in the first embodiment, even when a circuit that outputs a pulse signal is mounted on a chip, a chip area can be reduced.

### (Modification 2 of the First Embodiment)

A modification 2 of the first embodiment is explained.

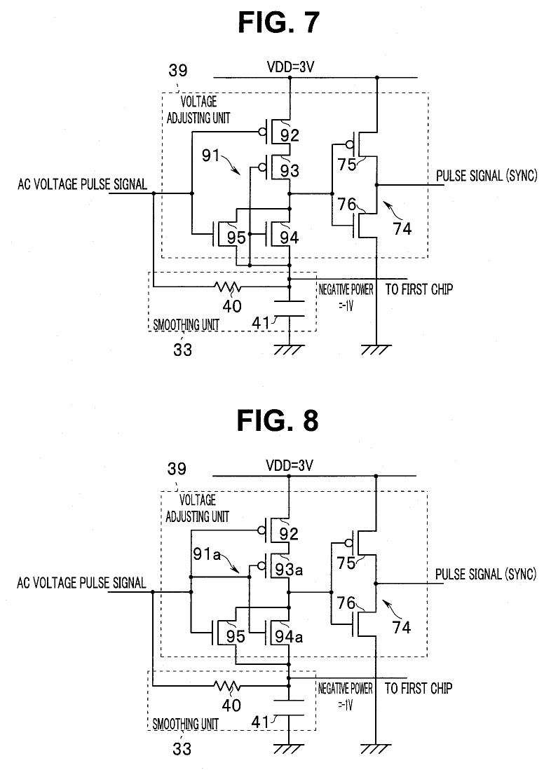

FIG. 7 is a circuit diagram showing another example of the configuration of the voltage adjusting unit 39. Note that, in FIG. 7, the same components as the components shown in FIG. 3 are denoted by the same reference numerals and signs and explanation of the components is omitted.

As shown in FIG. 7, the voltage adjusting unit 39 is configured by a NOR circuit 91 in a pre-stage and the inverter circuit 74 in the post-stage. The NOR circuit 91 is a two-input NOR gate and includes PMOS transistors 92 and 93 and NMOS transistors 94 and 95.

An AC voltage pulse signal is inputted to one input terminal of the NOR circuit 91 and an output (negative voltage=−1V) of the smoothing unit 33 is inputted to the other input terminal. More specifically, the AC voltage pulse signal is inputted to a gate terminal of the PMOS transistor 92 and a gate terminal of the NMOS transistor 95. The output (negative voltage=−1V) of the smoothing unit 33 is inputted to a gate terminal of the PMOS transistor 93 and a gate terminal of the NMOS transistor 94.

The NOR circuit 91 outputs an L signal when an H signal is inputted to at least one input terminal and outputs the H signal when other signals are inputted. Accordingly, when 1.5 V is inputted to one input terminal as the AC voltage pulse signal and the output (−1V) of the smoothing unit 33 is inputted to the other input terminal, the NOR circuit 91 outputs −1 V, which is the output of the smoothing unit 33. When −1 V is inputted to one input terminal as the AC voltage pulse signal and the output (−1V) of the smoothing unit 33 is inputted to the other input terminal, the NOR circuit 91 outputs 3 V.

The inverter circuit 74 in the post-stage has the same configuration as the configuration in the first embodiment. The inverter circuit 74 in the post-stage outputs an output signal of 0 V when an output signal of the NOR circuit 91 in the pre-stage is 3 V and outputs an output signal of 3 V when the output signal of the NOR circuit 91 in the pre-stage is −1 V. Consequently, as in the first embodiment, the voltage adjusting unit 39 can output a pulse signal, a High side of which is 3 V and a Low side of which is 0 V, to the timing generating unit 37.

In this way, the voltage adjusting unit 39 is configured by two logic circuits, that is, the NOR circuit 91 in the pre-stage and the inverter circuits 74 in the post-stage. Therefore, as in the first embodiment, a chip area can be set smaller than when the high-pass filter having the large time constant is provided.

FIG. 8 is a circuit diagram showing another example of the configuration of the voltage adjusting unit 39. Note that, in FIG. 8, the same components as the components shown in FIG. 7 are denoted by the same reference numerals and signs and explanation of the components is omitted.

As shown in FIG. 8, the voltage adjusting unit 39 is configured using a NOR circuit 91a instead of the NOR circuit 91 shown in FIG. 7. The NOR circuit 91a is configured using a PMOS transistor 93a and an NMOS transistor 94a respectively instead of the PMOS transistor 93 and the NMOS transistor 94 shown in FIG. 7.

An AC voltage pulse signal is inputted to a gate terminal of the PMOS transistor 93a and a gate terminal of the NMOS transistor 94a. The other components are the same as the components shown in FIG. 7. In other words, two input terminals of the NOR circuit 91a are shared. The AC voltage pulse signal is inputted to both of the two input terminals.

Accordingly, when 1.5 V is inputted to the two input terminals as the AC voltage pulse signal, the NOR circuit 91a outputs −1 V, which is an output of the smoothing unit 33. When −1 V is inputted to the two input terminals as the AC voltage pulse signal, the NOR circuit 91a outputs 3 V.

The inverter circuit 74 in the post-stage has the same configuration as the configuration in the first embodiment. The inverter circuit 74 in the post-stage outputs an output signal of 0 V when an output signal of the NOR circuit 91a in the pre-stage is 3 V and outputs an output signal of 3 V when the output signal of the NOR circuit 91a in the pre-stage is −1 V. Consequently, as in the first embodiment, the voltage adjusting unit 39 can output a pulse signal, a High side of which is 3 V and a Low side of which is 0 V, to the timing generating unit 37.

In this way, the voltage adjusting unit 39 is configured by two logic circuits, that is, the NOR circuit 91a in the pre-stage and the inverter circuit 74 in the post-stage. Therefore, as in the first embodiment, a chip area can be set smaller than when the high-pass filter having the large time constant is provided.

Accordingly, with the endoscope 2 functioning as an image pickup apparatus in the modification 2, as in the endoscope 2 in the first embodiment, even when a circuit that outputs a pulse signal is mounted on a chip, a chip area can be reduced.

### Second Embodiment

A second embodiment is explained.

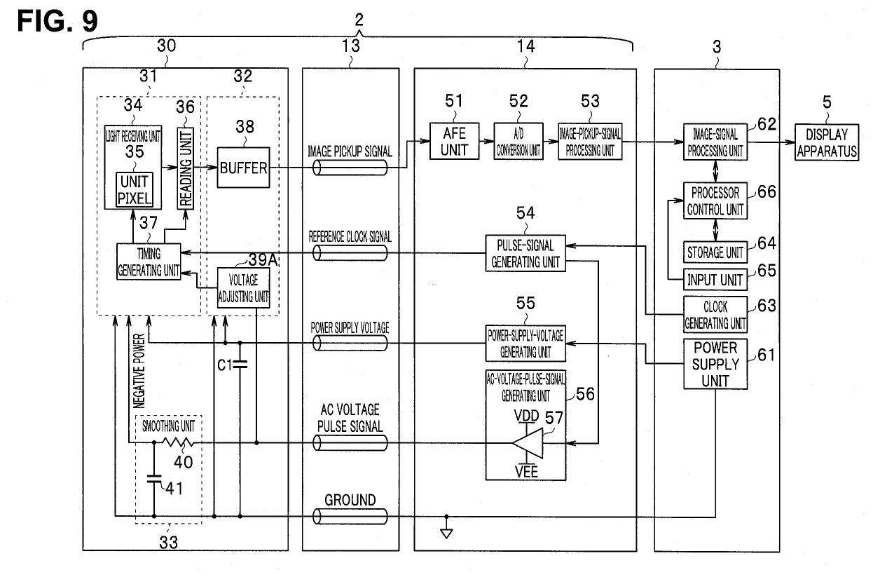

FIG. 9 is a block diagram showing a configuration of a main part of an endoscope system 1 in the second embodiment. Note that, in FIG. 9, the same components as the components shown in FIG. 2 are denoted by the same reference numerals and signs and explanation of the components is omitted.

As shown in FIG. 9, an image pickup unit 30 in the second embodiment is configured using a voltage adjusting unit 39A instead of the voltage adjusting unit 39 of the image pickup unit 30 shown in FIG. 2. The output of the smoothing unit 33 is connected to the voltage adjusting unit 39 shown in FIG. 2. In contrast, the output of the smoothing unit 33 is not connected to the voltage adjusting unit 39A in the present embodiment. In other words, only an AC voltage pulse signal from the AC-voltage-pulse-signal generating unit 56 is inputted to the voltage adjusting unit 39A in the present embodiment. The other components are the same as the components in the first embodiment.

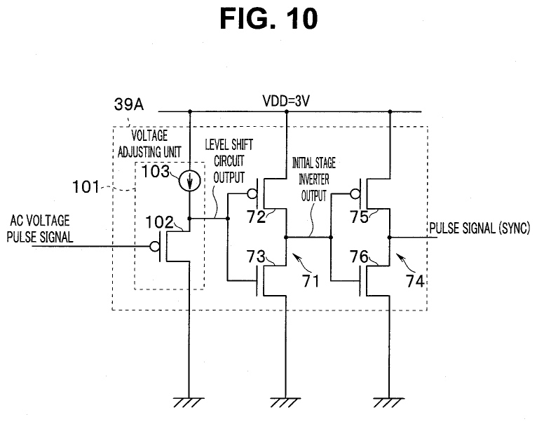

A detailed configuration of the voltage adjusting unit 39A is explained. FIG. 10 is a circuit configuration showing an example of a configuration of the voltage adjusting unit 39A. Note that, in FIG. 10, the same components as the components shown in FIG. 3 are denoted by the same reference numerals and signs and explanation of the components is omitted.

As shown in FIG. 10, the voltage adjusting unit 39A includes a level shift circuit 101, the inverter circuit 71 in the initial stage, and the inverter circuit 74 in the post-stage. The voltage adjusting unit 39A has a configuration in which the level shift circuit 101, the inverter circuit 71 in the initial stage, and the inverter circuit 74 in the post-stage are connected in series. An output terminal of the level shift circuit 101 is connected to an input terminal of the inverter circuit 71 in the initial stage. The output terminal of the inverter circuit 71 in the initial stage is connected to the input terminal of the inverter circuit 74 in the post-stage.

The level shift circuit 101 is configured by a source follower circuit configured by a PMOS transistor 102 and a constant current source 103. An AC voltage pulse signal from the AC-voltage-pulse-signal generating unit 56 is inputted to a gate terminal of the PMOS transistor 102. A source terminal of the PMOS transistor 102 is connected to the constant current source 103. A drain terminal of the PMOS transistor 102 is connected to the ground GND. In the present embodiment, the level shift circuit 101 shifts an inputted AC voltage pulse signal by +1 V and outputs the AC voltage pulse signal to the inverter circuit 71 in the initial stage.

In the first embodiment explained above, the source terminal of the NMOS transistor 73 of the inverter circuit 71 in the initial stage is connected to the output of the smoothing unit 33. In contrast, in the present embodiment, the source terminal of the NMOS transistor 73 of the inverter circuit 71 in the initial stage is connected to the ground GND. The other components are the same as the components in the first embodiment.

In this way, the voltage adjusting unit 39A in the present embodiment has a configuration in which the level shift circuit 101 and the inverter circuit 71 in the initial stage and the inverter circuit 74 in the post-stage, which are the logic circuits, are combined. The level shift circuit 101 is an analog circuit having an area larger than an area of the logic circuits. However, the level shift circuit 101 can be configured by a relatively simple circuit. An area of the level shift circuit 101 is small compared with the area of the high-pass filter having the large time constant. Accordingly, when the voltage adjusting unit 39A is incorporated in the second chip 32, an area of the second chip 32 can be reduced more than when the high-pass filter having the large time constant is incorporated in the second chip 32.

Operation of the endoscope system 1 in the second embodiment configured as explained above is explained.

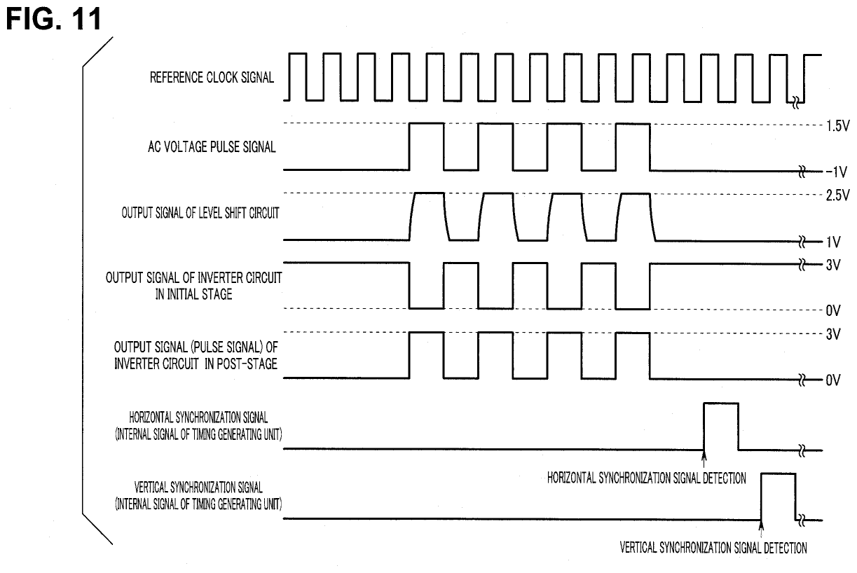

FIG. 11 is a timing chart showing an example of operation of the endoscope system according to the second embodiment. In FIG. 11, a reference clock signal, an AC voltage pulse signal, an output signal of the level shift circuit 101, an output signal of the inverter circuit 71 in the initial stage, an output signal of the inverter circuit 74 in the post-stage, a horizontal synchronization signal, and a vertical synchronization signal are shown in order from a top.

An AC voltage pulse signal, a High side of which is 4.5 V and a Low side of which is −1 V, is generated by the buffer amplifier 57 of the AC-voltage-pulse-signal generating unit 56. However, the High side of the AC voltage pulse signal outputted from the AC-voltage-pulse-signal generating unit 56 drops to 1.5 V because of attenuation in the universal cable 13. Accordingly, as shown in FIG. 11, an AC voltage pulse signal, a High side of which is 1.5 V and a Low side of which is −1 V, is inputted to the voltage adjusting unit 39A.

The level shift circuit 101 of the voltage adjusting unit 39A shifts the AC voltage pulse signal by +1 V and outputs the AC voltage pulse signal. More specifically, as shown in FIG. 11, the level shift circuit 101 outputs an output signal of 2.5 V to the inverter circuit 71 in the initial stage when the AC voltage pulse signal is 1.5 V. On the other hand, when the AC voltage pulse signal is −1 V, since 0 V of the ground GND is inputted to the level shift circuit 101, the level shift circuit 101 outputs an output signal of 1 V to the inverter circuit 71 in the initial stage.

A threshold of the inverter circuit 71 in the initial stage is 1.5 V. The source terminal of the PMOS transistor 72 of the inverter circuit 71 is connected to the power supply (VDD=3V) and the source terminal of the NMOS transistor 73 is connected to the ground circuit GND.

Accordingly, the inverter circuit 71 in the initial stage of the voltage adjusting unit 39A outputs an output signal of 0 V when an output signal of the level shift circuit 101 is 2.5 V and outputs an output signal of 3 V when the output signal of the level shift circuit 101 is 1 V.

A threshold of the inverter circuit 74 in the post-stage of the voltage adjusting unit 39A is 1.5 V. The source terminal of the PMOS transistor 75 of the inverter circuit 74 is connected to the power supply (VDD=3V) and the source terminal of the NMOS transistor 76 is connected to the ground GND (0V).

Accordingly, the inverter circuit 74 in the post-stage of the voltage adjusting unit 39A outputs an output signal of 0 V when an output signal of the inverter circuit 71 in the pre-stage is 3 V and outputs an output signal of 3 V when the output signal of the inverter circuit 71 in the pre-stage is 0 V.

Consequently, the voltage adjusting unit 39A outputs a pulse signal, a High side of which is 3 V and a Low side of which is 0 V, to the timing generating unit 37. The timing generating unit 37 generates a horizontal synchronization signal and a vertical synchronization signal based on the reference clock signal received from the pulse-signal generating unit 54 and the pulse signal received from the voltage adjusting unit 39A. The timing generating unit 37 generates a driving signal based on the generated horizontal synchronization signal and the generated vertical synchronization signal and outputs the driving signal to the light receiving unit 34 and the reading unit 36.

As explained above, the voltage adjusting unit 39A is configured by the level shift circuit 101 and the two inverter circuits 71 and 74, that is, three logic circuits. Therefore, when the voltage adjusting unit 39A is provided in the second chip 32, a chip area can be set smaller than when the high-pass filter having the large time constant is provided.

Accordingly, with the endoscope 2 functioning as the image pickup apparatus in the present embodiment, as in the endoscope 2 in the first embodiment, even when a circuit that outputs a pulse signal is mounted on a chip, a chip area can be reduced.

### Third Embodiment

A third embodiment is explained.

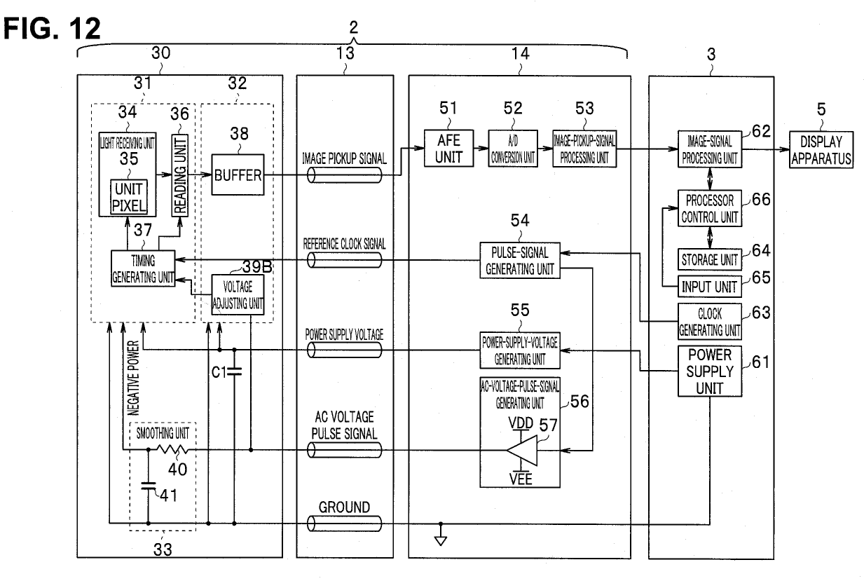

FIG. 12 is a block diagram showing a configuration of a main part of an endoscope system 1 in the third embodiment. Note that, in FIG. 12, the same components as the components shown in FIG. 9 are denoted by the same reference numerals and signs and explanation of the components is omitted.

As shown in FIG. 12, an image pickup unit 30 in the third embodiment is configured using a voltage adjusting unit 39B instead of the voltage adjusting unit 39A of the image pickup unit 30 shown in FIG. 9. The other components are the same as the components in the first embodiment.

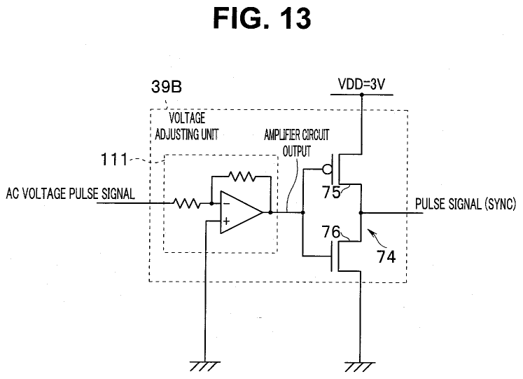

A detailed configuration of the voltage adjusting unit 39B is explained. FIG. 13 is a circuit diagram showing an example of a configuration of the voltage adjusting unit 39B. Note that, in FIG. 13, the same components as the components shown in FIG. 10 are denoted by the same reference numerals and signs and explanation of the components is omitted.

As shown in FIG. 13, the voltage adjusting unit 39B includes an amplifier circuit 111 and the inverter circuit 74 in the post-stage. The voltage adjusting unit 39B has a configuration in which the amplifier circuit 111 and the inverter circuit 74 in the post-stage are connected in series. An output terminal of the amplifier circuit 111 is connected to the input terminal of the inverter circuit 74 in the post-stage.

The amplifier circuit 111 is configured by an inverting amplifier circuit having a double gain. The amplifier circuit 111 multiplies an inputted AC voltage pulse signal by −2 and outputs the AC voltage pulse signal to the inverter circuit 74 in the post-stage. The other components are the same as the components in the first embodiment.

In this way, the voltage adjusting unit 39B in the present embodiment has a configuration in which the amplifier circuit 111 and the inverter circuit 74 in the post-stage, which is the logic circuit, are combined. The amplifier circuit 111 is an analog circuit having an area larger than the area of the logic circuit. However, the amplifier circuit 111 can be configured by a relatively simple circuit. The area of the amplifier circuit 111 is small compared with the high-pass filter having the large time constant. Accordingly, when the voltage adjusting unit 39B is incorporated in the second chip 32, an area of the second chip 32 can be reduced more than when the high-pass filter having the large time constant is incorporated in the second chip 32.

Operation of the endoscope system 1 in the third embodiment configured as explained above is explained.

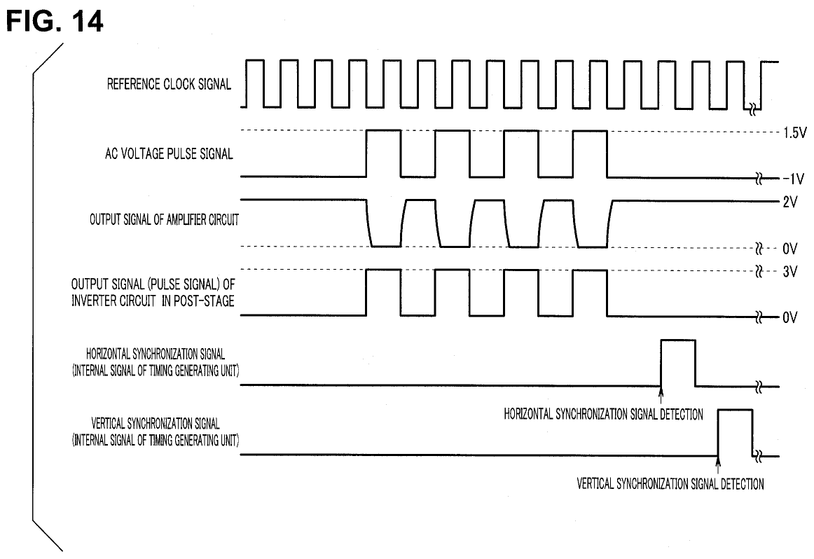

FIG. 14 is a timing chart showing an example of operation of the endoscope system according to the third embodiment. In FIG. 14, a reference clock signal, an AC voltage pulse signal, an output signal of the amplifier circuit 111, an output signal of the inverter circuit 74 in the post-stage, a horizontal synchronization signal, and a vertical synchronization signal are shown in order from a top.

An AC voltage pulse signal, a High side of which is 4.5 V and a Low side of which is −1 V, is generated by the buffer amplifier 57 of the AC-voltage-pulse-signal generating unit 56. However, the High side of the AC voltage pulse signal outputted from the AC-Voltage-pulse-signal generating unit 56 drops to 1.5 V because of attenuation in the universal cable 13. Accordingly, as shown in FIG. 14, an AC voltage pulse signal, a High side of which is 1.5 V and a Low side of which is −1 V, is inputted to the voltage adjusting unit 39B.

The amplifier circuit 111 of the voltage adjusting unit 39B inverts and amplifies the AC voltage pulse signal with a double gain and outputs the AC voltage pulse signal. As shown in FIG. 14, when the AC voltage pulse signal is 1.5 V and the amplifier circuit 111 inverts and amplifiers the AC voltage pulse signal with a double gain, the AC voltage pulse signal is −3 V. However, the amplifier circuit 111 outputs an output signal, an output voltage of which is a lower limit 0 V, to the inverter circuit 74 in the post-stage. On the other hand, when the AC voltage pulse signal is −1 V, the amplifier circuit 111 inverts and amplifies the AC voltage pulse signal with a double gain and outputs an output signal of 2 V to the inverter circuit 74 in the post-stage.

A threshold of the inverter circuit 74 in the post-stage of the voltage adjusting unit 39B is 1.5 V. The source terminal of the PMOS transistor 75 of the inverter circuit 74 is connected to the power supply (VDD=3V) and the source terminal of the NMOS transistor 76 is connected to the ground GND (0V).

Accordingly, the inverter circuit 74 in the post-stage of the voltage adjusting unit 39B outputs an output signal of 0 V when the output signal of the amplifier circuit 111 is 2 V and outputs an output signal of 3 V when the output signal of the amplifier circuit 111 is 0 V.

Consequently, the voltage adjusting unit 39B outputs a pulse signal, a High side of which is 3 V and a Low side of which is 0 V, to the timing generating unit 37. The timing generating unit 37 generates a horizontal synchronization signal and a vertical synchronization signal based on the reference clock signal received from the pulse-signal generating unit 54 and the pulse signal received from the voltage adjusting unit 39B. The timing generating unit 37 generates a driving signal based on the generated horizontal synchronization signal and the generated vertical synchronization signal and outputs the driving signal to the light receiving unit 34 and the reading unit 36.

As explained above, the voltage adjusting unit 39B is configured by the amplifier circuit 111 and the inverter circuit 74, that is, two logic circuits. Therefore, when the voltage adjusting unit 39B is provided in the second chip 32, a chip area can be set smaller than when the high-pass filter having the large time constant is provided.

Accordingly, with the endoscope 2 functioning as the image pickup apparatus in the present embodiment, as in the endoscope 2 in the first embodiment, even when a circuit that outputs a pulse signal is mounted on a chip, a chip area can be reduced.

The present invention is not limited to the embodiments explained above. Various changes, alterations, and the like are possible in a range in which the gist of the present invention is not changed.

What is claimed is:

1. An image pickup apparatus comprising:
an image pickup device including a plurality of pixels that are disposed in a two-dimensional matrix shape, the plurality of pixels being configured to receive light from an outside and generate an image pickup signal corresponding to a light reception amount;
a transmission cable for transmitting electric power to the image pickup device;
an AC-voltage-pulse-signal generation circuit provided on a proximal end side of the transmission cable, the AC-voltage-pulse-signal generation circuit being configured to generate an AC voltage pulse signal obtained by converting a positive voltage level and a negative voltage level of an inputted pulse signal into a predetermined positive voltage level and a predetermined negative voltage level, respectively, and output the AC voltage pulse signal to the transmission cable; and
a voltage adjustment circuit provided on a distal end side of the transmission cable, the voltage adjustment circuit being configured to convert the predetermined positive voltage level and the predetermined negative voltage level of the AC voltage pulse signal transmitted from the transmission cable into a DC voltage level and output a DC voltage pulse signal.

2. The image pickup apparatus according to claim 1, wherein
the AC-voltage-pulse-signal generation circuit includes a buffer amplifier driven by positive power having the predetermined positive voltage level and negative power having the predetermined negative voltage level, and
the buffer amplifier converts the positive voltage level of the inputted pulse signal into the predetermined positive voltage level with the positive power and converts the negative voltage level of the pulse signal into the predetermined negative voltage level with the negative power.

3. The image pickup apparatus according to claim 1, further comprising a smoothing circuit connected between the image pickup device and the transmission cable, the smoothing circuit being configured to generate a negative voltage from the AC voltage pulse signal transmitted from the transmission cable, wherein
the voltage adjustment circuit receives the negative voltage generated by the smoothing circuit and adjusts, using the negative voltage, the predetermined positive voltage level and the predetermined negative voltage level of the AC voltage pulse signal transmitted from the transmission cable.

4. The image pickup apparatus according to claim 3, wherein the voltage adjustment circuit has a configuration in which two or more logic circuits are combined and has a configuration in which a source terminal of an NMOS transistor of at least one logic circuit of the two or more logic circuits is connected to an output of the smoothing circuit and a source terminal of an NMOS transistor of the logic circuits excluding the at least one logic circuit is connected to a GND.

5. The image pickup apparatus according to claim 4, wherein the logic circuit has a configuration in which two or more inverter circuits are combined and has a configuration in which a source terminal of an NMOS transistor of an initial-stage inverter circuit is connected to the output of the smoothing circuit and a source terminal of an NMOS transistor of a post-stage inverter circuit is connected to the GND.

6. The image pickup apparatus according to claim 4, wherein the logic circuit has a configuration in which a NAND circuit and an inverter circuit or a NOR circuit and the inverter circuit are combined.

7. The image pickup apparatus according to claim 1, wherein the voltage adjustment circuit includes:
a level shift circuit configured to shift and output the predetermined positive voltage level and the predetermined negative voltage level of the AC voltage pulse signal transmitted from the transmission cable; and
an inverter circuit configured to invert and output an output of the level shift circuit.

8. The image pickup apparatus according to claim 1, wherein the voltage adjustment circuit includes:
an amplifier circuit configured to invert and amplify and output the AC voltage pulse signal transmitted from the transmission cable; and
an inverter circuit configured to invert and output an output of the amplifier circuit.

9. The image pickup apparatus according to claim 1, further comprising a timing generation circuit configured to generate, based on the DC voltage pulse signal outputted by the voltage adjustment circuit, a driving signal for driving the image pickup device.

10. An endoscope comprising:
an image pickup apparatus including:
an image pickup device including a plurality of pixels that are disposed in a two-dimensional matrix shape, the plurality of pixels being configured to receive light from an outside and generate an image pickup signal corresponding to a light reception amount;
a transmission cable for transmitting electric power to the image pickup device;
an AC-voltage-pulse-signal generation circuit provided on a proximal end side of the transmission cable, the AC-voltage-pulse-signal generation circuit being configured to generate an AC voltage pulse signal obtained by converting a positive voltage level and a negative voltage level of an inputted pulse signal into a predetermined positive voltage level and a predetermined negative voltage level, respectively, and output the AC voltage pulse signal to the transmission cable; and
a voltage adjustment circuit provided on a distal end side of the transmission cable, the voltage adjustment circuit being configured to convert the predetermined positive voltage level and the predetermined negative voltage level of the AC voltage pulse signal transmitted from the transmission cable into a DC voltage level and output a DC voltage pulse signal;
an insertion section insertable into a subject; and
a connector detachably attached to an image processing apparatus that applies image processing to the image pickup signal, wherein
the image pickup device and the voltage adjustment circuit are provided on a distal end side of the insertion section, and the AC-voltage-pulse-signal generation circuit is provided in the connector.

11. An endoscope system comprising:
an endoscope including:
an image pickup apparatus including:
an image pickup device including a plurality of pixels that are disposed in a two-dimensional matrix shape, the plurality of pixels being configured to receive light from an outside and generate an image pickup signal corresponding to a light reception amount;
a transmission cable for transmitting electric power to the image pickup device;
an AC-voltage-pulse-signal generation circuit provided on a proximal end side of the transmission cable, the AC-voltage-pulse-signal generation circuit being configured to generate an AC voltage pulse signal obtained by converting a positive voltage level and a negative voltage level of an inputted pulse signal into a predetermined positive voltage level and a predetermined negative voltage level, respectively, and output the AC voltage pulse signal to the transmission cable; and
a voltage adjustment circuit provided on a distal end side of the transmission cable, the voltage adjustment circuit being configured to convert the predetermined positive voltage level and the predetermined negative voltage level of the AC voltage pulse signal transmitted from the transmission cable into a DC voltage level and output a DC voltage pulse signal;
an insertion section insertable into a subject; and
a connector detachably attached to an image processing apparatus that applies image processing to the image pickup signal, wherein
the image pickup device and the voltage adjustment circuit are provided on a distal end side of the insertion section, and the AC-voltage-pulse-signal generation circuit is provided in the connector; and
the image processing apparatus configured to apply image processing to the image pickup signal.

12. A driving method for an image pickup apparatus configured from: an image pickup device including a plurality of pixels that are disposed in a two-dimensional matrix shape, the plurality of pixels being configured to receive light from an outside and generate an image pickup signal corresponding to a light reception amount; and a transmission cable for transmitting electric power to the image pickup device, the driving method for the image pickup apparatus comprising:
generating, with an AC-voltage-pulse-signal generation circuit provided on a proximal end side of the transmission cable, an AC voltage pulse signal obtained by converting a positive voltage level and a negative voltage level of an inputted pulse signal into a predetermined positive voltage level and a predetermined negative voltage level, respectively, and outputting the AC voltage pulse signal to the transmission cable;
converting, with a voltage adjustment circuit provided on a distal end side of the transmission cable, the predetermined positive voltage level and the predetermined negative voltage level of the AC voltage pulse signal transmitted from the transmission cable into a DC voltage level and outputting a DC voltage pulse signal; and
driving the image pickup device using the DC voltage pulse signal.
